(19) United States
(12) Patent Application Publication
(10) Pub. No.: US 2025/0241608 A1
(43) Pub. Date: Jul. 31, 2025

(54) METHOD FOR OPERATING AN X-RAY APPARATUS, X-RAY APPARATUS, COMPUTING DEVICE, COMPUTER PROGRAM AND ELECTRONICALLY READABLE DATA STORAGE MEDIUM

(71) Applicant: Siemens Healthineers AG, Forchheim (DE)

(72) Inventors: Alois Regensburger, Poxdorf (DE); Bernd Niedermeyer, Effeltrich (DE)

(21) Appl. No.: 19/037,277

(22) Filed: Jan. 26, 2025

(30) Foreign Application Priority Data
Jan. 26, 2024 (DE) 10 2024 200 705.6

(51) Int. Cl. A61B 6/00

(52) U.S. Cl. CPC A61B 6/5223; A61B 6/54

(57) ABSTRACT

A method for operating an X-ray apparatus includes acquiring at least one 2D X-ray image of an object under examination by an X-ray device and 2D/3D registering the at least one 2D X-ray image with a 3D model by a computing device of the X-ray apparatus. 2D image elements of the at least one 2D X-ray image are associated with corresponding 3D image elements that are situated at respective 3D image-element locations in the 3D model. A volume of interest is determined within the 3D model, and the at least one 2D X-ray image is post-processed. At least one of the 2D image elements is edited according to a relative location, in relation to the volume of interest, of the 3D image-element location of the 3D image element corresponding to the at least one 2D image element. Output data including the at least one 2D X-ray image is provided.

This application claims the benefit of German Patent Application No. DE 10 2024 200 705.6, filed on Jan. 26, 2024, which is hereby incorporated by reference in its entirety.

### BACKGROUND

The present embodiments relate to operating an X-ray apparatus.

In certain minimally invasive intervention techniques, X-ray images are created in order to guide an instrument into the region to be treated in the patient. In the case of vertebroplasty, kyphoplasty, or more broadly defined procedures in which the instrument is guided into/along a bone or another high-contrast anatomical structure, certain bone structures captured in the X-ray images may have an adverse effect on image quality in a region of interest because they make it harder to visually identify important landmarks of a bone (e.g., of a vertebral body) that are relevant to the treatment. These additional distracting bone structures lie, in a projection direction of the X-ray images, in front of and/or behind the region of interest relevant to carrying out the procedure.

Typical X-ray images acquired during vertebroplasty show a multiplicity of anatomical, and in some cases artificial, structures from different depth planes that are depicted simultaneously superimposed on one another. These superimposed structures often lie precisely in the region of interest in which an instrument tip and its location in relation to a relevant anatomy is meant to be identified by the X-ray images.

Two-dimensional (2D) bone removal methods in which bones are identified in the X-ray images and completely removed from the X-ray images are known from the prior art. This would not be helpful in the aforementioned treatment techniques because relevant bone landmarks are precisely what are meant to remain in the X-ray images.

Virtual bokeh methods or virtual depth-of-focus methods are used in smartphone cameras in order to accentuate in images regions lying at a certain depth in comparison with regions at other depths. In this case, however, the structures do not lie on top of each other in the image. In addition, the depth information is typically obtained using multi-view geometry using a plurality of sensors. Therefore, the aforementioned methods cannot be applied to improving X-ray images.

The following methods are known for improving X-ray images.

U.S. Pat. No. 11,195,309 B2 discloses a method and a system for generating X-ray images of an object. According to the present embodiments, a shift-and-add method is used for generating a stack of linear tomography planes each associated with a different region inside the object. A set of shift values is defined from the consideration of providing that the stack of linear tomography planes fills in the tomographic volume with a spatial density adequate to the application. If so required by the application, some focal planes may be selectively processed for sharpness reduction in some regions and to control the depth-of-field. A focus stacking method is used to synthesize a single 2D X-ray image from the tomographic stack of images. A depth map of in-focus regions from the linear tomography stack may be used for creating a 3D object model.

US 2017/0281110 A1 discloses a method for identifying a tomographic image from a number of tomographic images. The method includes receiving information specifying a region of interest in at least one of a number of projection images or in at least one of a number of tomographic images reconstructed from the plurality of projection images. The method includes identifying a tomographic image of the number of tomographic images, where the identified tomographic image is in greater focus in an area corresponding to the region of interest than others of the number of tomographic images.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a representation of structures of a region of interest in X-ray images is improved.

A first aspect of the present embodiments relates to a method for operating an X-ray apparatus. The X-ray apparatus has an X-ray device and a computing device.

The method includes acquiring at least one two-dimensional (2D) X-ray image of an object under examination by the X-ray device of the X-ray apparatus. The X-ray device may have a flat-panel detector, for example, that is configured to capture X-ray radiation that is emitted by an X-ray source and passes through the object under examination arranged between the X-ray source and the flat-panel detector.

A subsequent act of the method includes 2D/three-dimensional (3D) registration of the at least one 2D X-ray image with a 3D model by the computing device of the X-ray apparatus. In other words, the computing device performs the 2D/3D registration, in which the 2D X-ray image is associated with the 3D model. The 3D model may be created, for example, from 2D X-ray images from a previous acquisition of the object under examination, and may depict the object under examination in three dimensions. Alternatively, the 3D model may represent a generic object. In the 2D/3D registration, 2D image elements of the at least one 2D X-ray image are associated with respective corresponding 3D image elements of the 3D model. The 3D image elements are situated at respective 3D image-element locations in the 3D model. In other words, the at least one 2D X-ray image includes the 2D image elements. The 2D image elements form the 3D image elements of the 3D model.

The image elements may be, for example, anatomical features (e.g., bones, organs, or blood vessels). It is also possible that the image elements are, for example, medical instruments (e.g., guide wires, catheters, endoscopes, or surgical instruments). In addition, the image elements may be, for example, implants (e.g., screws, spine reinforcements, or artificial joints). For example, the image elements may be represented as image regions having high image values (e.g., pixel values or voxel values), as edges, or as contours.

The 3D image elements may be situated at different 3D image-element locations in the 3D model. In the case that the 3D model is a three-dimensional depiction of the object under examination, the fact that the 2D X-ray image is a projection of the object under examination provides that 2D image-element locations of the respective 2D image elements in the particular 2D X-ray image depend on the 3D image-element locations of the respective corresponding 3D image elements. If the 3D model represents a generic object, the 2D image-element locations correspond to respective 3D image-element locations. If two of the 3D image elements are situated one behind the other with respect to a projection direction of the 2D X-ray image concerned, the two 3D image elements are situated at different depths. If the two 3D image elements are not offset with respect to one other along a direction perpendicular to the projection direction, the corresponding 2D image elements are depicted on top of each other at an identical 2D image-element location.

In other words, the 2D image elements in the 2D X-ray image may be superimposed, at least in part. For example, it may be the case that the 2D X-ray image represents the 2D image elements associated with 3D image elements located one behind the other along the projection path. The corresponding 3D image elements may thus contribute jointly to a value of an image point of the 2D X-ray image.

In a further act of the method, a volume of interest within the 3D model is determined. In other words, a corresponding volume of the 3D model is depicted by the at least one 2D X-ray image. It is provided that a certain volume of interest of the volume of the 3D model is meant to be accentuated over the rest of the volume. The volume of interest concerned is determined for the 3D model.

A further act of the method includes post-processing the at least one X-ray image. In the post-processing, one or more of the 2D image elements are edited according to a relative location, in relation to the volume of interest, of the 3D image-element location of the 3D image elements corresponding to the respective 2D image element(s). In other words, the 2D image elements that represent the 3D image elements of the 3D model are depicted by the at least one 2D X-ray image. Some of the 3D image elements are located inside the volume of interest of the 3D model, and others of the 3D image elements are located outside the volume of interest. The editing of image elements is achieved by editing the image values (e.g., the pixel values) belonging to the particular image element. The pixel values of image elements may be increased or decreased, for example, or edges or contours may be accentuated to a greater or lesser degree by raising or lowering contrasts. The editing of the individual 2D image elements depends on whether their associated 3D image element lies inside or outside the volume of interest. For example, it may be provided that for the 2D image elements for which the corresponding 3D image element lies inside the volume of interest, a different editing method is applied than for the 2D image elements for which the associated 3D image element lies outside the volume of interest. For example, it may be provided that 2D image elements, the 3D image elements of which lie inside the volume of interest, are accentuated in the at least one 2D X-ray image by the editing, and that 2D image elements for which the corresponding 3D image element lies outside the volume of interest are attenuated or filtered out in the 2D X-ray image.

In a further act, output data is provided by the computing device. The output data includes the at least one edited 2D X-ray image.

The present embodiments have the advantage that an X-ray image in which 2D image elements that correspond to 3D image elements inside a volume of interest are especially accentuated may be provided.

One development of the present embodiments provides that the post-processing of the at least one 2D X-ray image includes editing, according to a first editing rule, one or more of the 2D image elements for which the corresponding 3D image element has a 3D image-element location situated inside the volume of interest. In other words, a first editing rule is specified for 2D image elements that are projections of corresponding 3D image elements inside the volume of interest. The first editing rule may specify, for example, that the computing device adjusts contrasts of the 2D image elements concerned. This may accentuate the 2D image elements associated with the volume of interest.

One development provides that the post-processing of the at least one 2D X-ray image includes editing, according to a second editing rule, one or more of the 2D image elements for which the corresponding 3D image element has a 3D image-element location situated outside the volume of interest. In other words, the second editing rule is specified to the computing device in order to edit the 2D image elements that are projections of a 3D image element outside the volume of interest. For example, it may be provided that the second editing rule provides filtering out, blurring, or contrast-reduction of the relevant 2D image elements. This may attenuate 2D image elements associated with 3D image elements from other volumes.

One development of the present embodiments provides that the post-processing of the at least one 2D X-ray image includes editing, according to a third editing rule, one or more of the 2D image elements for which the corresponding 3D image element is identified as a specified object. In other words, the third editing rule may be assigned to the 2D image elements that are associated with corresponding 3D image elements of a specified object of the 3D model. 2D image elements associated with the specified object are edited by the computing device in accordance with the third editing rule. It may be provided, for example, that the specified object describes an instrument or an anatomical anomaly. It may be provided that the 2D image elements associated with the object shall be edited in accordance with the third editing rule regardless of whether the object is situated inside or outside the volume of interest. This may allow certain objects to be depicted or hidden regardless of whether the objects are situated inside or outside the volume of interest. For example, the specified object may be specified manually or identified by the computing devices.

One development of the present embodiments provides that the post-processing of the at least one 2D X-ray image includes determining an image point of the 2D X-ray image at which a plurality of the 2D image elements are superimposed. In other words, an image point may be located in a region of the 2D X-ray image at which 2D image elements are superimposed. The superimposed 2D image elements may correspond to 3D image elements located one behind the other along the projection direction and thus located in different depth planes.

The image point may have a total value that represents the absorption of an X-ray beam along the projection direction. The X-ray concerned may be absorbed by tissue or material of the respective objects associated with the 3D image elements. The 2D image elements may show, for example, bones located one behind the other. The total value of the image point is formed by partial values of the respective 2D image elements.

In other words, the total value is obtained from the partial values of the respective 2D image elements.

It is provided that the editing of the partial values of the respective 2D image elements is carried out in accordance with the respective editing rules specified for the 2D image elements. For example, it may be the case that two 2D image elements are superimposed at the image point. The two 2D image elements may be assigned different depth information.

It may consequently be provided that one of the 2D image elements is to be edited in accordance with the first editing rule, and the other of the 2D image elements is to be edited in accordance with the second editing rule. Since the image point depicts both of the 2D image elements, it may be necessary to edit the corresponding partial values separately. For example, it may be possible that one of the 2D image elements is not meant to be modified in accordance with the first editing rule. Consequently, the partial value originating from the 2D image element concerned is not modified. It may be provided that the second 2D image element is meant to be filtered out in accordance with the second editing rule. Consequently, the partial value originating from the 2D image element concerned is removed. After the editing, the total value of the image point thus equals only the partial value of one of the 2D image elements. The development has the advantage of allowing a particular pixel to be edited.

One development of the present embodiments provides that the method includes determining, in the 3D model, a reference object location of a specified reference object depicted in the at least one 2D X-ray image. In other words, a reference object depicted in the 2D X-ray image is specified. The reference object may be, for example, an instrument or an anatomical structure. The reference object may be selected in the 2D X-ray image according to a user input. It may also be provided that the reference object is identified automatically by the computing device, or the reference object location of the reference object in the 3D model is provided by an external tracking system. For example, it may be provided that the reference object location of the reference object is determined by an optical navigation system and/or by a surgical navigation system and provided to the computing device. The computing device determines the reference object location of the depicted reference object in the 3D model.

A further act of the method includes determining the volume of interest within the 3D model according to the reference object location of the reference object. In other words, the volume of interest within the 3D model is determined by the computing device according to the reference object location of the reference object. It may be provided, for example, that the volume of interest is defined, according to a rule, around the reference object location of the reference object. It may be provided, for example, that the reference object describes an instrument. The volume of interest may have specified dimensions and be disposed around a tip of the instrument as the center point. This has the advantage that the volume of interest may be selected automatically based on the reference object. Thus, when guiding the instrument through the object, there is no need to track the location of the volume of interest manually because the location of the volume of interest is coupled to the reference object location of the instrument.

One development of the present embodiments provides that the method includes receiving a spatial path in relation to the 3D model for guiding a reference object through the 3D model. In other words, the path of the reference object, along which the reference object is guided through the 3D model, is made available to the computing unit. For example, the path may describe a trajectory along which a tip of an instrument is meant to be guided through the 3D model.

The method includes determining the volume of interest within the 3D model according to the spatial course of the spatial path through the 3D model. In other words, it is provided that the volume of interest is selected by the computing devices according to the course of the path through the 3D model. For example, it may be provided that the volume of interest describes a volume around the path or a certain segment of the path of the reference object through the 3D model. This has the advantage that the region relevant to guiding the reference object may be accentuated.

One development of the present embodiments provides that the method includes receiving a workflow in relation to the 3D model by the computing device. In other words, the computing device is provided with the workflow relating to handling of the 3D model.

The method includes determining the volume of interest within the 3D model according to a current status of the workflow. In other words, the volume of interest within the 3D model is defined by the computing unit according to the current status of the workflow.

For cases of use or situations of use that may arise in the method and are not explicitly described here, it may be provided that, according to the method, an error message and/or a prompt to enter user feedback is output and/or a default setting and/or a predefined initial state is set.

A second aspect of the present embodiments relates to an X-ray apparatus that has an X-ray device and a computing device.

The X-ray device is configured to acquire at least one 2D X-ray image of an object under examination. The computing device is configured to perform 2D/3D registration of the at least one 2D X-ray image with a 3D model, where 2D image elements of the at least one 2D X-ray image are associated with corresponding 3D image elements that are situated at respective 3D image-element locations in the 3D model. The computing device is configured to determine a volume of interest within the 3D model.

The computing device is configured to post-process the at least one 2D X-ray image, where at least some of the 2D image elements are edited according to a relative location, in relation to the volume of interest, of the 3D image-element location of the 3D image elements corresponding to the respective 2D image elements. The computing device is configured to provide output data including the at least one 2D X-ray image.

A third aspect of the present embodiments relates to a computing device.

The computing device is configured to perform 2D/3D registration of at least one 2D X-ray image with a 3D model, where 2D image elements of the at least one 2D X-ray image are associated with corresponding 3D image elements that are situated at respective 3D image-element locations in the 3D model. The computing device is configured to determine a volume of interest within the 3D model.

The computing device is configured to post-process the at least one 2D X-ray image, where at least some of the 2D image elements are edited according to a relative location, in relation to the volume of interest, of the 3D image-element location of the 3D image elements corresponding to the respective 2D image elements. The computing device is configured to provide output data including the at least one 2D X-ray image.

The aforementioned object is also achieved according to the present embodiments by a computer program that may be loaded directly into a memory of a computing device, and has program means in order to perform the acts of the aforementioned method according to the second aspect of the present embodiments when the program is executed in the computing device.

Likewise, there may be an electronically readable data storage medium (e.g., a non-transitory computer-readable storage medium) having electronically readable control information stored thereon. The electronically readable control information includes at least one computer program (product) described and is configured such that the at least one computer program performs the described method according to the first aspect of the present embodiments when the data storage medium is used in a computing device.

The advantages and developments presented above in connection with the method according to the present embodiments according to the first aspect apply mutatis mutandis also to the X-ray apparatus according to the present embodiments, to the computing device according to the present embodiments, to the computer program according to the present embodiments, and to the electronically readable data storage medium according to the present embodiments. The presented method features of the method may accordingly be regarded as features of the X-ray apparatus, of the computing device, of the computer program, and of the electronically readable data storage medium.

The storage medium may include a storage unit.

A computing unit may be understood to be, for example, a data processing unit that contains a processing circuit. For example, the computing unit may thus process data for performing computing operations. These include operations for performing indexed accesses to a data structure (e.g., to a look-up table (LUT)).

The computing unit may contain, for example, one or more computers, one or more microcontrollers, and/or one or more integrated circuits (e.g., one or more application-specific integrated circuits (ASICs), one or more field-programmable gate arrays (FPGAs), and/or one or more systems on a chip (SoCs)). The computing unit may also contain one or more processors (e.g., one or more microprocessors, one or more central processing units (CPUs), one or more graphics processing units (GPUs), and/or one or more signal processors, such as one or more digital signal processors (DSPs)). The computing unit may also contain a physical or virtual interconnection of computers or other of the aforementioned units.

In various example embodiments, the computing unit contains one or more hardware and/or software interfaces and/or one or more memory units.

A memory unit may be configured as a volatile data storage medium (e.g., as a dynamic random access memory (DRAM) or a static random access memory (SRAM)) or as a non-volatile data storage medium (e.g., as a read-only memory (ROM), as a programmable read-only memory (PROM), as an erasable programmable read-only memory (EPROM), as an electrically erasable programmable read-only memory (EEPROM), as a flash memory or flash EEPROM, as a ferroelectric random access memory (FRAM), as a magnetoresistive random access memory (MRAM), or as a phase-change random access memory (PCRAM)).

Independent of the grammatical term usage, individuals with male, female, or other gender identities are included within the term.

The claims, the figures, and the description of the figures contain further features of the invention. The features and feature combinations mentioned above in the description, and the features and feature combinations mentioned below in the description of the figures and/or shown in the figures may be included by the invention not just in the particular combination stated but also in other combinations. For example, the invention may include embodiments and feature combinations that do not have all the features of a claim in the original wording. Further, the invention may include embodiments and feature combinations that go beyond or differ from the feature combinations presented in the dependency references of the claims.

The invention is described in greater detail below with reference to specific example embodiments and associated schematic drawings. In the figures, same or functionally same elements may be denoted by the same reference characters. The description of same or functionally same elements is not necessarily repeated when referring to different figures.

### BRIEF DESCRIPTION OF THE DRAWINGS

FIG. 1 shows a schematic representation of an X-ray apparatus;

FIG. 2 shows a schematic representation of a flow of a method for operating an X-ray apparatus; and

FIG. 3 shows a schematic representation of a 3D model of an object under examination.

### DETAILED DESCRIPTION

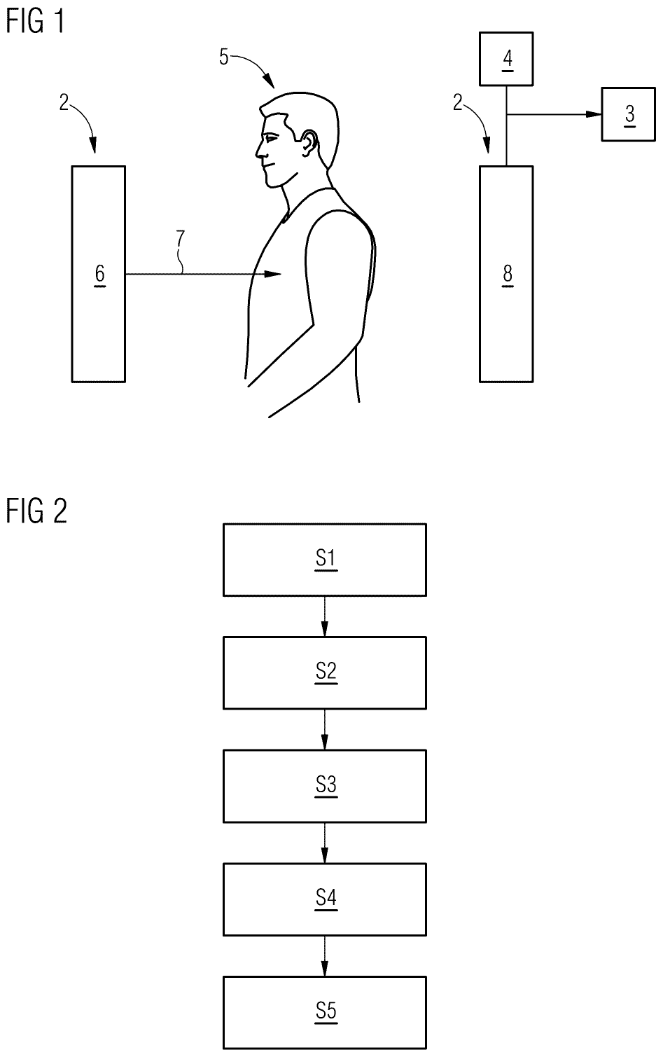

FIG. 1 shows a schematic representation of an X-ray apparatus.

The X-ray apparatus 1 may have an X-ray device 2 and a computing device 3. The X-ray apparatus 1 may be intended for acquiring X-ray images 4 of an object under examination 5. The X-ray device 2 may have an X-ray source 6 that may be configured to emit X-ray radiation along a projection direction 7. The projection direction 7 may be oriented towards a flat-panel detector 8 of the X-ray device 2. The object under examination 5 may be located between the X-ray source 6 and the flat-panel detector 8. The object under examination 5 may have locally different absorption properties. The computing device 3 may be intended to read out raw data from the flat-panel detector 8 and to generate the X-ray images 4.

FIG. 2 shows a schematic representation of a flow of a method for operating an X-ray apparatus.

The method may be performed, for example, by the X-ray apparatus 1 shown in FIG. 1.

In act S1, at least one 2D X-ray image 4 of an object under examination 5 may be acquired by an X-ray device 2 of the X-ray apparatus 1.

In act S2, 2D/3D registration of the at least one 2D X-ray image 12 with a 3D model 9 may be performed by a computing device of the X-ray apparatus. In the act, 2D image elements 13 of the at least one 2D X-ray image 12 may be associated with corresponding 3D image elements 10 that are situated at respective 3D image-element locations in the 3D model 9.

In act S3, a volume of interest 14 within the 3D model 9 may be determined by the computing device 2. It may be provided, for example, that the determining of the volume of interest 14 includes identifying a reference object 11 in the at least one 2D X-ray image 12, and subsequently determining a reference location of the reference object 11 within the 3D model 9. The reference object 11 may be an instrument, for example.

In act S4, post-processing of the at least one 2D X-ray image 12 may be performed by the computing device 2. In this process, at least some of the 2D image elements 13 are edited according to a relative location, in relation to the volume of interest 14, of the 3D image-element location of the 3D image elements 10 corresponding to the respective 2D image elements 13. For example, it may be provided that at least some of the 2D image elements 13 for which the corresponding 3D image elements 10 lie inside the volume of interest 14 are edited according to a first editing rule by the computing device 2. The first editing rule may include, for example, image processing steps that improve visibility of the 2D image elements 13 in the at least one 2D X-ray image 12. For example, the first editing rule may stipulate an increase in contrast, sharpening, and/or marking of the 2D image elements 13 concerned. At least some of the 2D image elements 13 for which the corresponding 3D image elements 10 lie outside the volume of interest 14 may be edited according to a second editing rule by the computing device 2. The second editing rule may be intended to attenuate the 2D image elements 13 concerned in the at least one 2D X-ray image 12. For example, it may be provided that a sharpness of the relevant 2D image elements 13 is reduced and/or contrast values of the relevant 2D image elements 13 are lowered. This may accentuate the 2D image elements 13 inside the volume of interest 14 even more clearly over 2D image elements 13 outside the volume of interest 14.

It may be provided that 2D image elements 13 for which the corresponding 3D image element 10 is identified as a specified object 15 are edited according to a third editing rule by the computing device 2. This may be carried out regardless of the 3D image-element location of the object 15 concerned. In other words, it is irrelevant whether the object 15 lies inside or outside the volume of interest 14. It may be provided, for example, that the 2D image element 13 concerned is accentuated in accordance with the third editing rule even if the 2D image element 13 is situated outside the volume of interest 14. This may be provided, for example, if the object 15 relates to defined anomalies identified by the computing device 2 or to specific instruments. It may also be provided to filter out certain 2D image elements 13 inside the volume of interest 14 that depict the specified object 15.

In a fifth act S5, output data may be provided by the computing device 2. The output data includes the at least one processed 2D X-ray image 17.

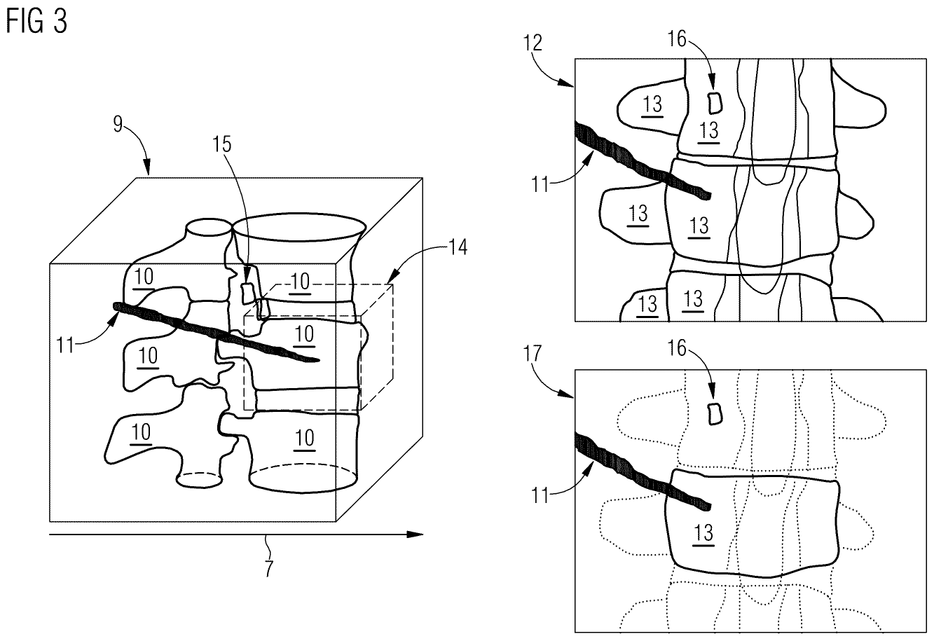

FIG. 3 shows a schematic representation of a 3D model of an object under examination.

The 3D model 9 of the object under examination 5 may be generated, for example, from 2D X-ray images of the object under examination 5 from a previous examination. The 3D model 9 may depict 3D image elements 10. For example, the 3D image elements 10 may be anatomical structures such as bones. The 3D image elements 10 may also include a reference object 11. The reference object 11 may be an instrument. FIG. 3 also shows an unprocessed 2D X-ray image 12 that may be acquired from the object under examination 5 by the X-ray apparatus 1. The unprocessed 2D X-ray image 12 may be acquired along a projection direction that is shown in relation to the 3D model 9. In the unprocessed 2D X-ray image 12, 2D image elements 13 may be depicted that may correspond to 3D elements 10 of the 3D model 9. The 2D image elements 13 may be superimposed in the unprocessed 2D X-ray image 12.

It may be required that certain of the 2D image elements 13 are accentuated over others of the 2D image elements 13. This may be made dependent on whether the associated 3D image elements 10 of the 2D image elements 13 are situated inside or outside a volume of interest 14. The volume of interest 14 may define a volume that is meant to be accentuated in a processed 2D X-ray image 17. For example, the volume of interest 14 may depend on a reference location of the reference object 11. It may be provided, for example, that the volume of interest 14 describes a region around a tip of a reference object 11 in the form of an instrument.

A first editing rule may be stored in the computing device 3. The first editing rule may specify the manner in which the 2D image elements 13, the 3D image elements 10 of which are located inside the volume of interest 14, are meant to be edited. The first editing rule may be intended to accentuate the relevant 2D image elements 13. A second editing rule may be stored in the computing device 3. The second editing rule relates to 2D image elements 13, the 3D image elements 10 of which may be located outside the volume of interest 14. The second editing rule may be aimed at attenuating the relevant 2D image elements 13. A third editing rule may also be stored in the computing device 3. The third editing rule may specify how 2D image elements 13 associated with specified objects 15 (e.g., the reference object 11 or an anomaly) are meant to be edited. It may be provided, for example, that the 2D image elements 13 of the relevant objects 15 are accentuated in accordance with the third editing rule even if the relevant objects 15 are located outside the volume of interest 14. Equally, it may be provided that the 2D image elements 13 of the relevant objects 15 are filtered out in accordance with the third editing rule even if the 2D image elements 13 of the relevant objects 15 are located inside the volume of interest 14. The third editing rule may hence be prioritized over the first editing rule and/or the second editing rule. The processed X-ray image 17 is also shown, which may be generated by the computing device 3 by processing the unprocessed X-ray image 12.

Depicted in the processed X-ray image 17 is one of the 2D image elements 13 for which the associated 3D image element 10 is situated inside the volume of interest 14 and is therefore accentuated in the processed X-ray image 17 in accordance with the first editing rule. The 2D image elements 13 for which the associated 3D image elements 10 are located outside the volume of interest 14 may be presented attenuated in accordance with the second editing rule. The relevant 3D image elements 10 may lie in front of or behind the volume of interest 14 along the projection direction 7, for example. The 2D image element 13 inside the volume of interest 14 is hence accentuated in comparison with the unprocessed 2D X-ray image 12, so that an operator may be assisted in guiding the reference object 11 into the volume of interest 14. An anomaly visible in the 3D model 9 may be identified by the computing device 3 and defined as one of the objects 15. For example, the object 15 may be a splitter. In the edited X-ray image 17, a 2D image element 16 associated with the splitter may be accentuated in accordance with the third editing rule even though the 2D image element 16 associated with the splitter is located outside the volume of interest 14. It is thereby possible to accentuate anomalies in the edited X-ray 17.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for operating an X-ray apparatus, the method comprising:
acquiring at least one two-dimensional (2D) X-ray image of an object under examination by an X-ray device of the X-ray apparatus;
2D/three-dimensional (3D) registering the at least one 2D X-ray image with a 3D model by a computing device of the X-ray apparatus, wherein 2D image elements of the at least one 2D X-ray image are associated with corresponding 3D image elements that are situated at respective 3D image-element locations in the 3D model;
determining a volume of interest within the 3D model;
post-processing the at least one 2D X-ray image, wherein at least one of the 2D image elements is edited according to a relative location, in relation to the volume of interest, of the 3D image-element location of the 3D image element corresponding to the at least one 2D image element; and
providing output data comprising the at least one 2D X-ray image.

2. The method of claim 1, wherein the post-processing of the at least one 2D X-ray image comprises:
editing, according to a first editing rule, at least one of the 2D image elements for which the corresponding 3D image element has a 3D image-element location situated inside the volume of interest.

3. The method of claim 2, wherein the post-processing of the at least one 2D X-ray image comprises:
editing, according to a second editing rule, at least one of the 2D image elements for which the corresponding 3D image element has a 3D image-element location situated outside the volume of interest.

4. The method of claim 3, wherein the post-processing of the at least one 2D X-ray image comprises:
editing, according to a third editing rule, at least one of the 2D image elements for which the corresponding 3D image element is identified as a specified object.

5. The method of claim 1, wherein the post-processing of the at least one 2D X-ray image comprises:
determining an image point of the 2D X-ray image at which a plurality of the 2D image elements are superimposed, wherein partial values of the respective 2D image elements of the image point form a total value of the image point; and
editing the partial values of the respective 2D image elements in accordance with the respective editing rules specified for the 2D image elements.

6. The method of claim 1, further comprising:
determining, in the 3D model, a reference object location of a specified reference object depicted in the at least one 2D X-ray image; and
determining the volume of interest within the 3D model according to the reference object location of the reference object in the 3D model.

7. The method of claim 1, further comprising:
receiving a spatial path in relation to the 3D model for guiding a reference object through the 3D model; and
determining the volume of interest within the 3D model according to a spatial course of the spatial path through the 3D model.

8. The method of claim 1, further comprising:
receiving a workflow in relation to the 3D model; and
determining the volume of interest within the 3D model according to a current status of the workflow.

9. An X-ray apparatus comprising:
an X-ray device; and
a computing device,
wherein the X-ray device is configured to acquire at least one two-dimensional (2D) X-ray image of an object under examination,
wherein the computing device is configured to:
perform 2D/three-dimensional (3D) registration of the at least one 2D X-ray image with a 3D model, wherein 2D image elements of the at least one 2D X-ray image are associated with corresponding 3D image elements that are situated at respective 3D image-element locations in the 3D model;
determine a volume of interest within the 3D model;
post-process the at least one 2D X-ray image, wherein at least one of the 2D image elements is edited according to a relative location, in relation to the volume of interest, of the 3D image-element location of the 3D image element corresponding to the at least one 2D image element; and
provide output data comprising the at least one 2D X-ray image.

10. A computing device comprising:
a processor configured to:
perform two-dimensional (2D)/three-dimensional (3D) registration of at least one 2D X-ray image with a 3D model, wherein 2D image elements of the at least one 2D X-ray image are associated with corresponding 3D image elements that are situated at respective 3D image-element locations in the 3D model;
determine a volume of interest within the 3D model;
post-process the at least one 2D X-ray image, wherein at least one of the 2D image elements is edited according to a relative location, in relation to the volume of interest, of the 3D image-element location of the 3D image element corresponding to the at least one 2D image element; and
provide output data comprising the at least one 2D X-ray image.

11. In a non-transitory computer-readable storage medium that stores instructions executable by one or more processors to operate an X-ray apparatus, the instructions comprising:
acquiring at least one two-dimensional (2D) X-ray image of an object under examination by an X-ray device of the X-ray apparatus;
2D/three-dimensional (3D) registering the at least one 2D X-ray image with a 3D model by a computing device of the X-ray apparatus, wherein 2D image elements of the at least one 2D X-ray image are associated with corresponding 3D image elements that are situated at respective 3D image-element locations in the 3D model;
determining a volume of interest within the 3D model;
post-processing the at least one 2D X-ray image, wherein at least one of the 2D image elements is edited according to a relative location, in relation to the volume of interest, of the 3D image-element location of the 3D image element corresponding to the at least one 2D image element; and
providing output data comprising the at least one 2D X-ray image.

12. The non-transitory computer-readable storage medium of claim 11, wherein the post-processing of the at least one 2D X-ray image comprises:
editing, according to a first editing rule, at least one of the 2D image elements for which the corresponding 3D image element has a 3D image-element location situated inside the volume of interest.

13. The non-transitory computer-readable storage medium of claim 12, wherein the post-processing of the at least one 2D X-ray image comprises:
editing, according to a second editing rule, at least one of the 2D image elements for which the corresponding 3D image element has a 3D image-element location situated outside the volume of interest.

14. The non-transitory computer-readable storage medium of claim 13, wherein the post-processing of the at least one 2D X-ray image comprises:
editing, according to a third editing rule, at least one of the 2D image elements for which the corresponding 3D image element is identified as a specified object.

15. The non-transitory computer-readable storage medium of claim 11, wherein the post-processing of the at least one 2D X-ray image comprises:
determining an image point of the 2D X-ray image at which a plurality of the 2D image elements are superimposed, wherein partial values of the respective 2D image elements of the image point form a total value of the image point; and
editing the partial values of the respective 2D image elements in accordance with the respective editing rules specified for the 2D image elements.

16. The non-transitory computer-readable storage medium of claim 11, wherein the instructions further comprise:
determining, in the 3D model, a reference object location of a specified reference object depicted in the at least one 2D X-ray image; and
determining the volume of interest within the 3D model according to the reference object location of the reference object in the 3D model.

17. The non-transitory computer-readable storage medium of claim 11, wherein the instructions further comprise:
receiving a spatial path in relation to the 3D model for guiding a reference object through the 3D model; and
determining the volume of interest within the 3D model according to a spatial course of the spatial path through the 3D model.

18. The non-transitory computer-readable storage medium of claim 11, wherein the instructions further comprise:
receiving a workflow in relation to the 3D model; and
determining the volume of interest within the 3D model according to a current status of the workflow.
